(19) United States
(12) Patent Application Publication
(10) Pub. No.: US 2024/0076405 A1
(43) Pub. Date: Mar. 7, 2024

(54) EXPRESSION VECTORS FOR IMMUNOGLOBULINS AND APPLICATIONS THEREOF

(71) Applicant: Canton Biologics Co., Ltd., Guangzhou (CN)

(72) Inventors: Qianhui LIANG, Guangzhou (CN); Yang LIU, Guangzhou (CN); Dongxue FEI, Guangzhou (CN); Jana FRANK, Guangzhou (CN)

(21) Appl. No.: 18/494,117

(22) Filed: Oct. 25, 2023

(30) Foreign Application Priority Data
Aug. 26, 2022 (CN) 202211032991.2

(51) Int. Cl. C07K 16/32; C12N 15/85

(52) U.S. Cl. CPC C07K 16/32; C12N 15/85

(57) ABSTRACT

The present disclosure provides an expression vector, comprising: a first expression cassette having a first transcriptional regulatory element and an immunoglobulin heavy chain-coding region operatively linked to the first transcriptional regulatory element; a second expression cassette having a second transcriptional regulatory element and an immunoglobulin light chain-coding region operatively linked to the second transcriptional regulatory element; and a third expression cassette having a third transcriptional regulatory element and an immunoglobulin J chain-coding region operatively linked to the third transcriptional regulatory element. The present disclosure further relates to the host cell comprising the expression vector and the use thereof.

### INCORPORATION OF SEQUENCE LISTING

This application contains a sequence listing submitted in Computer Readable Form (CRF). The CFR file contains the sequence listing entitled “SequenctListing.xml”, which was created on Oct. 24, 2023, and is 23,157 bytes in size. The information in the sequence listing is incorporated herein by reference in its entirety.

### TECHNICAL FIELD

The present disclosure relates to the field of biotechnology, in particular to expression vectors and expression systems for expressing immunoglobulins, as well as the uses thereof.

### BACKGROUND

Due to the high specificity of immunoglobulins, especially monoclonal or polyclonal antibodies, they have been widely used in many fields such as cell biology, basic medicine, clinical diagnostics and therapeutics.

An immunoglobulin contains heavy and light chains. IgA or IgM further contains a J chain. The transcription level of each chain may strongly affect the expression level thereof finally affect the expression level of the antibody proteins and the survival rate of cells. Moreover, IgM has a higher molecular weight and a lower secretion efficiency for the multimers. It leads to endoplasmic reticulum (ER) stress, thereby dramatically deteriorating its yields.

Therefore, there is a need to develop a vector that can effectively express antibodies to realize higher yields and minimizing the impact on host cells.

### SUMMARY OF THE INVENTION

In order to solve at least one of the above technical problems in the art, the present disclosure provides an expression vector for immunoglobulins, particularly an expression vector for IgA or IgM which has a J chain, and the use thereof.

According to one aspect, the present disclosure provides an expression vector which comprises: a first expression cassette having a first transcriptional regulatory element and an immunoglobulin heavy chain (HC)-coding region operatively linked to the first transcriptional regulatory element; a second expression cassette having a second transcriptional regulatory element and an immunoglobulin light chain (LC)-coding region operatively linked to the second transcriptional regulatory element; and a third expression cassette having a third transcriptional regulatory element and an immunoglobulin J chain-coding region operatively linked to the third transcriptional regulatory element. In some embodiments, the expression level(s) of the first and/or second expression cassette are not less than that of the third expression cassette. That is, the expression levels of the first and/or second expression cassette, at the transcript or protein level, are not less than that of the third expression cassette, i.e., the expression level(s) of the first and/or second expression cassette the expression level of the third expression cassette. In some embodiments, the first expression cassette, the second expression cassette and the third expression cassette are located within the same vector.

In some embodiments, the first and/or second transcriptional regulatory elements may have a capability of initiating transcription higher than that of the third transcriptional regulatory element. The first and/or second transcriptional regulatory elements may have higher affinity to RNA polymerase than that of the third promoter, thereby enabling the gene operatively connected therewith to have a higher level of expression.

In some embodiments, the first transcriptional regulatory element may comprise a first promoter.

In some embodiments, the second transcriptional regulatory element may comprise a second promoter.

In some embodiments, the third transcriptional regulatory element may comprise a third promoter.

In some embodiments, the first promoter, the second promoter and/or the third promoter may be promoters for expression in mammalian cells. The promoter for expression in mammalian cells is well known by one skilled in the art and generally comprises those capable of inducing the expression of a gene in mammalian cells. In some embodiments, the first promoter, the second promoter and/or the third promoter may be one or more selected from a group consisting of of CMV promoter, EF1α promoter, CAG promoter, CBH promoter, SFFV promoter, EFS promoter, MSCV promoter, SV40 promoter, mPGK promoter, hPGK promoter, UBC promoter, human beta actin promoter, TRE promoter, UAS promoter, ac5 promoter, Polyhedrin promoter, CaMKIIa promoter and an artificial promoter.

In some embodiments, the first promoter and/or the second promoter may be one or more selected from a group consisting of of CMV promoter, EF1α promoter, CAG promoter, CBH promoter, human beta actin promoter, SFFV promoter and an artificial promoter. In some embodiments, the first promoter may be located upstream of the immunoglobulin heavy chain-coding region. In some embodiments, the second promoter may be located upstream of the immunoglobulin light chain-coding region.

In some embodiments, the first promoter and/or the second promoter may contain promoters having higher transcription-initiating capability, such as one or more selected from a group consisting of of CMV promoter, EF1α promoter, CAG promoter, CBH promoter, human beta actin promoter, SFFV promoter and an artificial promoter.

In some embodiments, the third promoter may be one or more selected from a group consisting of of CMV promoter, EF1α promoter, CAG promoter, CBH promoter, SFFV promoter, EFS promoter, MSCV promoter, SV40 promoter, mPGK promoter, hPGK promoter, UBC promoter, human beta actin promoter, TRE promoter, UAS promoter, AC5 promoter, Polyhedrin promoter and CaMKIIa promoter. In some embodiments, the third promoter may contain promoters having lower transcription-initiating capability, such as SV40 promoter, UBC promoter, EFS promoter, MSCV promoter, mPGK promoter and hPGK promoter.

In some embodiments, the third promoter may be located upstream of the immunoglobulin J chain-coding region.

In some embodiments, the third promoter may contain CMV promoter.

In some embodiments, the CMV promoter may be derived from human CMV (hCMV) promoter or mouse CMV (mCMV) promoter. In some embodiments, the CMV promoter may contain a nucleic acid sequence as shown in SEQ ID NO: 1, or a nucleic acid sequence having at least 80%, 85%, 90%, 95%, 96%, 97%, 98%, or 99% sequence identity to the nucleic acid sequence as shown in SEQ ID NO: 1.

In some embodiments, the EF1α promoter may contain a nucleic acid sequence as shown in SEQ ID NO: 2, or a nucleic acid sequence having at least 80%, 85%, 90%, 95%, 96%, 97%, 98%, or 99% sequence identity to the nucleic acid sequence as shown in SEQ ID NO: 2.

In some embodiments, the first transcriptional regulatory element and/or the second transcriptional regulatory element may further contain another transcriptional regulatory element, such as an intron, an enhancer and the like. In some embodiments, the first transcriptional regulatory element and/or the second transcriptional regulatory element may further contain one or more intron(s). In some embodiments, the intron may be located downstream of the first promoter and/or the second promoter. In some embodiments, the intron may be located upstream of the immunoglobulin heavy chain-coding region. In some embodiments, the intron may be located upstream the immunoglobulin light chain-coding region.

In some embodiments, the intron may be selected from a group consisting of a human β-globin intron, a CMV intron or an EF1α intron.

In some embodiments, the intron may be selected from a group consisting of human β-globin intron 2, CMV intron 1 or EF1α intron A.

In some embodiments, the intron may be selected from human β-globin introns. In preferred embodiments, the intron may contain a nucleic acid sequence as shown in SEQ ID NO: 3, or a nucleic acid sequence having at least 80%, 85%, 90%, 95%, 96%, 97%, 98%, or 99% sequence identity to the nucleic acid sequence as shown in SEQ ID NO: 3.

In some embodiments, the intron may be selected from CMV introns. In preferred embodiments, the intron may contain a nucleic acid sequence as shown in SEQ ID NO: 4, or a nucleic acid sequence having at least 80%, 85%, 90%, 95%, 96%, 97%, 98%, or 99% sequence identity to the nucleic acid sequence as shown in SEQ ID NO: 4.

In some embodiments, the intron may be selected from EF1α introns. In preferred embodiments, the intron may contain a nucleic acid sequence as shown in SEQ ID NO: 5, or a nucleic acid sequence having at least 80%, 85%, 90%, 95%, 96%, 97%, 98%, or 99% sequence identity to the nucleic acid sequence as shown in SEQ ID NO: 5.

In some embodiments, the immunoglobulin may include IgM and IgA. In specific embodiments, the immunoglobulin may be IgM. In some embodiments, the immunoglobulin may be any type of antibodies, such as murine, chimeric, humanized, and human antibodies.

IgM, when containing a J chain, may form stable pentamers or hexamers. The J chain also plays important roles in the mucosal transport of IgM mediated by the polymeric immunoglobulin receptor (pIgR). However, IgM generally has a molecular weight greater than 900 kDa and the secretion of its multimer is lowered. It leads to ER sress, thereby dramatically deteriorating its yields. In the present disclosure, it is unexpectedly found that the aforementioned combinations of the transcriptional regulatory elements, particularly in one vector, can realize higher levels of the expression of heavy and light chains than that of J chain, which results in a higher level of the expression of the immunoglobulins (e.g., IgM). For example, the immunoglobulins (e.g., IgM) may have a yield of 5-6 g/L in a cell pool, even up to 8 g/L in monoclonal cells.

In the present disclosure, an expression vector is constructed by combining the expression cassettes for a light chain, a heavy chain, and a J chain of IgM in the same vector. Meanwhile, the transcription regulatory elements are incorporated at the 5′-terminals of the light chain, the heavy chain, and the J chain, to modulate the expression or transcription ratios of the light chain, the heavy chain, and the J chain. When the expression level(s) of the light chain and/or heavy chain are higher than that of the J chain (i.e., light chain/heavy chain: J chain≥1), the level of the expression of IgM can be increased, and the corresponding regulatory elements are the preferred regulatory elements. The regulatory element(s) for the light and heavy chains may comprise a promoter, a combination of a promoter and an intron, or a combination of a promoter and an enhancer. The transcriptional regulatory elements for the light and heavy chains may comprise a promoter having a high capability of initiating transcription (i.e., a strong promoter), a combination of a strong promoter and an intron, or a combination of a strong promoter and an enhancer. The transcriptional regulatory element of the J chain may comprise a strong promoter or a promoter having a moderate capability of initiating transcription, or comprise a promoter having a low capability of initiating transcription (i.e., a weak promoter) and/or an intron.

In some embodiments, the enhancer may be derived from any eukaryotic organism, including but not limited to, for example, an enhancer located on the late side of SV40 replication origin (100-270 bp), cytomegalovirus (CMV) immediate-early enhancer, an enhancer located on the late side of polyoncovirus replication origin, and an enhancer from the adenovirus.

In some embodiments, an expression vector may comprise: a first expression cassette having a first CMV promoter and an immunoglobulin heavy chain-coding region operatively linked to the first CMV promoter; a second expression cassette having a second CMV promoter and an immunoglobulin light chain-coding region operatively linked to the second CMV promoter; and, a third expression cassette having a third SV40 promoter and an immunoglobulin J chain-coding region operatively linked to the third SV40 promoter, wherein the expression levels of the first expression cassette and/or the second expression cassette are higher than that of the third expression cassette, and wherein the first expression cassette, the second expression cassette and the third expression cassette are located within the same vector.

In some embodiments, an expression vector may comprise: a first expression cassette having a first CMV promoter and an immunoglobulin heavy chain-coding region operatively linked to the first CMV promoter; a second expression cassette having a second CMV promoter and an immunoglobulin light chain-coding region operatively linked to the second CMV promoter; and a third expression cassette having a third UBC promoter and an immunoglobulin J chain-coding region operatively linked to the UBC third promoter, wherein the expression levels of the first expression cassette and/or the second expression cassette are higher than that of the third expression cassette, and wherein the first expression cassette, the second expression cassette and the third expression cassette are located within the same vector. In some embodiments, the third expression cassette may optionally further contain an intron of EF1α.

In some embodiments, an expression vector may comprise: a first expression cassette having a first promoter, an intron of EF1α, and an immunoglobulin heavy chain-coding region operatively linked to the first CMV promoter; a second expression cassette having a second CMV promoter, an intron of EF1α, and an immunoglobulin light chain-coding region operatively linked to the second CMV promoter; and a third expression cassette having a third CMV promoter and an immunoglobulin J chain-coding region operatively linked to the third CMV promoter, wherein the expression levels of the first expression cassette and/or the second expression cassette are higher than that of the third expression cassette, and wherein the first expression cassette, the second expression cassette and the third expression cassette are located within the same vector. In some embodiments, the third expression cassette may optionally further contain an intron of EF1α.

In some embodiments, an expression vector may comprise: a first expression cassette having a first CMV promoter, an intron of EF1α, and an immunoglobulin heavy chain-coding region operatively linked to the first CMV promoter; a second expression cassette having a second CMV promoter, an intron of EF1α, and an immunoglobulin light chain-coding region operatively linked to the second CMV promoter; and a third expression cassette having a third SV40 promoter, and an immunoglobulin J chain-coding region operatively linked to the third SV40promoter, wherein the expression levels of the first expression cassette and/or the second expression cassette are higher than that of the third expression cassette, and wherein the first expression cassette, the second expression cassette and the third expression cassette are located within the same vector. In some embodiments, the third expression cassette may optionally further contain an intron of EF1α.

In some embodiments, an expression vector may comprise: a first expression cassette having a first CMV promoter, a CMV enhancer, and an immunoglobulin heavy chain-coding region operatively linked to the first CMV promoter; a second expression cassette having a second CMV promoter, a CMV enhancer, and an immunoglobulin light chain-coding region operatively linked to the second CMV promoter; and a third expression cassette having a third CMV promoter and an immunoglobulin J chain-coding region operatively linked to the third CMV promoter, wherein the expression levels of the first expression cassette and/or the second expression cassette are higher than that of the third expression cassette, and wherein the first expression cassette, the second expression cassette, and the third expression cassette are located within the same vector.

In some embodiments, an expression vector may comprise: a first expression cassette having a first EF1α promoter, an intron of EF1α, and an immunoglobulin heavy chain-coding region operatively linked to the first EF1α promoter; a second expression cassette having a second EF1α promoter, an intron of EF1α, and an immunoglobulin light chain-coding region operatively linked to the second EF1α promoter; and a third expression cassette having a third SV40 promoter, and an immunoglobulin J chain-coding region operatively linked to the third SV40promoter, wherein the expression levels of the first expression cassette and/or the second expression cassette are higher than that of the third expression cassette, and wherein the first expression cassette, the second expression cassette and the third expression cassette are located within the same vector.

In some embodiments, an expression vector may comprise: a first expression cassette having a first EF1α promoter, an intron of CMV, and an immunoglobulin heavy chain-coding region operatively linked to the first EF1α promoter; a second expression cassette having a second EF1α promoter, an intron of CMV, and an immunoglobulin light chain-coding region operatively linked to the second EF1α promoter; and a third expression cassette having a third SV40 promoter, and an immunoglobulin J chain-coding region operatively linked to the third EF1α promoter, wherein the expression levels of the first expression cassette and/or the second expression cassette are higher than that of the third expression cassette, and wherein the first expression cassette, the second expression cassette and the third expression cassette are located within the same vector.

In some embodiments, an expression vector may comprise: a first expression cassette having a first CAG promoter, an intron of CMV, and an immunoglobulin heavy chain-coding region operatively linked to the first CAG promoter; a second expression cassette having a second CAG promoter, an intron of CMV, and an immunoglobulin light chain-coding region operatively linked to the second CAG promoter; and a third expression cassette having a third SV40 promoter, and an immunoglobulin J chain-coding region operatively linked to the third SV40 promoter, wherein the expression levels of the first expression cassette and/or the second expression cassette are higher than that of the third expression cassette, and wherein the first expression cassette, the second expression cassette and the third expression cassette are located within the same vector.

In some embodiments, the ratio of the expression levels of the immunoglobulin heavy chain, the immunoglobulin light chain and the J chain may be (4-20):(4-20):(1-4) at the transcript or protein level.

In some embodiments, the ratio of the expression levels of the immunoglobulin heavy chain, the immunoglobulin light chain and the J chain may be (4-10):(4-15):(1-4) at the transcript or protein level.

In some embodiments, the expression level of the immunoglobulin heavy chain, at the transcript or protein level, may be higher than or equal to the expression level of the immunoglobulin light chain. In some embodiments, the ratio of the expression level of the immunoglobulin heavy chain to that of the immunoglobulin light chain is 0.1-10 at the transcript or protein level. In some embodiments, the ratio of the expression level of the immunoglobulin heavy chain to that of the immunoglobulin light chain may be in a range of about 0.4, 0.5, 0.6, 0.7, 0.8, 0.9, 1.0, 1.1, 1.2, 1.3, 1.4, 1.5, 1.6, 1.7, 1.8, 1.9 or 2.0 at the transcript or protein level.

The present disclosure comprises a method for expressing a protein using the vector described herein. Therefore, the present disclosure provides a method for producing a recombinant protein, which comprises the following steps: introducing the expression vector of the present disclosure into a mammalian host cell; culturing the mammalian host cells under a condition suitable for the expression of the protein; and, harvesting the protein. The advantage of the vector of the present disclosure at least lies in the high yield of the protein by means of a mammalian cell culture system.

According to another aspect, the present disclosure provides a host cell comprising the expression vector of the present disclosure.

Any type of cells, which is capable of realizing gene expression by using the expression vector disclosed herein, can be used as the host cell of the present disclosure. The term “host cell” refers to a cell that has been transformed with the vector which is constructed through recombinant DNA technology.

Those skilled in the art can select specific host cell lines that are suitable for antibody expression through the expression vectors disclosed herein. The host cells that can be used in the present disclosure may include mammalian cells, cell lines, and cell cultures derived therefrom. Mammalian cells (e.g., germ cells or somatic cells) may be derived from mammals, such as rodents including mouse, rat or the like, or primates including humans, monkeys or the like. Both primary cells and immortalized cells are suitable for antibody expression via transformation with the expression vector disclosed herein.

In some embodiments, the host cell may be derived from mammals, including but not limited to Chinese hamster ovary (CHO) cells, Chinese hamster fibroblast (e.g., R1610 cell), human cervical cancer cells (e.g., HeLa cell), monkey kidney cells (e.g., CVI and COS cells), murine fibroblast (e.g., BALBc/3T3 cell), murine myeloma cells (P3.times.63-Ag3.653; NS0; SP2/O cell), hamster kidney cells (e.g., HAK cell), murine L cells (e.g., L-929 cell), human lymphocytes (e.g., RAJI cell), human embryonic kidney cells (e.g., 293 and 293T cell), GEX® cell line, myeloid leukemia cells (e.g., cell lines derived from GT-5 cells). In some embodiments, the host cell may be Chinese hamster ovary (CHO) cells.

The expression vector disclosed herein can be introduced or transformed into the host cell through any technique known in the art.

According to yet another aspect, the present disclosure provides a method for producing an immunoglobulin, which comprises the following steps: culturing the host cell of the present disclosure; and harvesting the immunoglobulin. In some embodiments, the immunoglobulin may comprise IgM or IgA, preferably is IgM.

According to yet another aspect, the present disclosure provides an expression vector, which comprises: a first expression cassette having a first transcriptional regulatory element and an immunoglobulin heavy chain-coding region operatively linked to the first transcriptional regulatory element; a second expression cassette having a second transcriptional regulatory element and an immunoglobulin light chain-coding region operatively linked to the second transcriptional regulatory element; and a third expression cassette having a third transcriptional regulatory element and an immunoglobulin J chain-coding region operatively linked to the third transcriptional regulatory element, wherein the first transcriptional regulatory element and/or the second transcriptional regulatory element has a capability of initiating transcription no less than that of the third transcriptional regulatory element, and wherein the first expression cassette, the second expression cassette and the third expression cassette are located within the same vector.

According to yet another aspect, the present disclosure provides a method for increasing an expression level of an immunoglobulin, wherein the ratio of the expression level of an immunoglobulin heavy chain and/or light chain to that of the J chain is greater than 1, and the ratio of the expression level of the heavy chain to that of the light chain is in a range of 0.1 to 10.

In some embodiments, the ratio of the expression level of the heavy chain to that of J chain may be in a range of 1 to 20. In some embodiments, the ratio of the expression level of the heavy chain to that of J chain may be in a range of 1 to 15. In some embodiments, the ratio of the expression level of the heavy chain to that of J chain may be about 1, 2, 3, 4, 5, 6, 7, 8, 9, 10, 11, 12, 13, 14 or 15.

In some embodiments, the ratio of the expression level of the light chain to that of J chain may be in a range of 1 to 20. In some embodiments, the ratio of the expression level of the light chain to that of J chain may be in a range of 1 to 15. In some embodiments, the ratio of the expression level of the light chain to that of J chain may be about 1, 2, 3, 4, 5, 6, 7, 8, 9, 10, 11, 12, 13, 14 or 15.

In some embodiments, the ratio of the expression level of the heavy chain to that of the light chain may be in a range of 0.125 to 8.0. In some embodiments, the ratio of the expression level of the heavy chain to that of the light chain may be in a range of 0.2 to 5.0. In some embodiments, the ratio of the expression level of the heavy chain to that of the light chain is in a range of 0.3 to 3.0. In some embodiments, the ratio of the expression level of the heavy chain to that of the light chain may be in a range of 0.4 to 2.0. In some embodiments, the ratio of the expression level of the heavy chain to that of the light chain may be about 0.4, 0.5, 0.6, 0.7, 0.8, 0.9, 1.0, 1.1, 1.2, 1.3, 1.4, 1.5, 1.6, 1.7, 1.8, 1.9 or 2.0.

In some embodiments, the immunoglobulin may comprise IgM and/or IgA. In specific embodiments, the immunoglobulin may be IgM.

IgM is a class of immunoglobulins with the largest molecular weight. IgM which contains an J chain forms a pentamer, and IgM which contains no J chain forms a hexamer. Currently, the yield of IgM-pentamers is very low, especially in mammalian cells (e.g., CHO cells), with a yield of generally about 100-300 mg/L. Surprisingly, the present disclosure can achieve an extremely high yield of IgM in mammalian cells by combining the heavy-, light- and J chains-coding sequences of IgM into the same vector and using the specific combinations of transcriptional regulatory elements having different transcription-initiating capabilities, so as to modulate the ratio of the expression levels of the heavy-, light- and J chains.

### BRIEF DESCRIPTION OF THE DRAWINGS

FIG. 1A shows the schematic diagram of the vector p3MGT-IgM-1 which is established according to one embodiment of the present disclosure.

FIG. 1B shows the schematic diagram of the vector p3MGT-IgM-2 which is established according to one embodiment of the present disclosure.

FIG. 2A shows the structural schematic diagram of the vector p2MGT-HC-LC which is established according to one embodiment of the present disclosure.

FIG. 2B shows the structural schematic diagram of the vector pMGT-JC which is established according to one embodiment of the present disclosure.

FIG. 3 shows the viable cell densities according to one embodiment of the present disclosure.

FIG. 4 shows the cell viability rates according to one embodiment of the present disclosure.

FIG. 5 shows the SDS-PAGE electrophoresis results of IgMs expressed in various ways according to one embodiment of the present disclosure, wherein Lane M: 10-200 kD ladder; Lane 1: CD20 p3MGT-IgM-1 (non-reduced); Lane 2: CD20 p2MGT-HC-LC:CD20 pMGT-JC=5:1 (non-reduced); Lane 3: CD20 p2MGT-HC-LC:CD20 pMGT-JC=4:1 (non-reduced); Lane 4: CD20 p3MGT-IgM-1 (reduced); Lane 5: CD20 p2MGT-HC-LC:CD20 pMGT-JC=5:1 (reduced); Lane 6: CD20 p2MGT-HC-LC:CD20 pMGT-JC=4:1 (reduced); HC: 64.4 kD; LC: 25.4 kD; JC: 17.2 kD (J chain appears as a band about 25 kD in SDS-PAGE).

FIG. 6 shows the non-denatured PAGE results of anti-CD20 IgM expressed according to one embodiment of the present disclosure. Lane 1: CD20 p2MGT-HC-LC:CD20 pMGT-JC=5:1; Lane 2: CD20 p2MGT-HC-LC:CD20 pMGT-JC=4:1; Lane 3: CD20 p3MGT-IgM-1; Lane 4: CD20 p3MGT-IgM-2.

FIG. 7 shows the denatured SDS-PAGE results of anti-CD20 IgM expressed through different vector systems according to one embodiment of the present disclosure. Lane 1: CD20 p2MGT-HC-LC:CD20 pMGT-JC=5:1 (reduced); Lane 2: CD20 p2MGT-HC-LC:CD20 pMGT-JC=4:1 (reduced); Lane 3: CD20 p3MGT-IgM-1 (reduced); Lane 4: CD20 p3MGT-IgM-2 (reduced).

FIGS. 8A-8D show the transcription levels of HC, LC, and JC of anti-CD20 IgM expressed through different vectors according to one embodiment of the present disclosure. FIG. 8A: CD20 p2MGT-HC-LC:CD20 pMGT-JC=5:1; FIG. 8B: CD20 p2MGT-HC-LC:CD20 pMGT-JC=4:1; FIG. 8C: CD20 p3MGT-IgM-1; FIG. 8D: CD20 p3MGT-IgM-2.

FIG. 9 shows the viable cell densities and viability rates according to one embodiment of the present disclosure.

FIG. 10 shows the expression results of anti-Her2 IgM antibody according to one embodiment of the present disclosure. Wherein Lane 1: Her2 p3MGT-IgM-1 (reduced); Lane 2: Her2 p3MGT-IgM-1 (non-reduced); Lane 3: Her2 p2MGT-HC-LC:Her2 pMGT-JC=5:1 (reduced); Lane 4: Her2 p2MGT-HC-LC:Her2 pMGT-JC=5:1 (non-reduced); Lane 5: Her2 p2MGT-HC-LC:Her2 pMGT-JC=4:1 (reduced); Lane 6: Her2 p2MGT-HC-LC:Her2 pMGT-JC=4:1 (non-reduced). HC chain is about 64.4 kD; LC chain is about 25.4 kD; and JC chain is about 17.2 kD and appears as a band about 25 kD in SDS-PAGE.

### DETAILED DESCRIPTION OF THE INVENTION

The following examples are merely provided to facilitate understanding the present invention. However, it should be understood that these examples are only intended to illustrate the present invention and not to constitute any limitations. The actual scope of protection of the present invention is defined in the claims. It should be further understood that any modifications and changes may be made without departing from the spirit of the present invention.

Unless otherwise defined, all technical terms and scientific terms used in the present invention have the same meanings as those commonly used in the field to which the present invention belongs. For the purpose of interpreting the present description, the following definitions will apply, and where appropriate, the terms used in singular will also include their plural forms, and vice versa.

Unless otherwise explicitly stated in the context, the expressions “a” and “an” used herein include plural references. For example, referring to “a cell” includes multiple cells and other equivalents as known to those skilled in the art.

As used herein, the term “a nucleic acid” or “a nucleic acid molecule” is intended to include DNA, RNA, mRNA, cDNA, genomic DNA, and analogs thereof. A nucleic acid molecule may be single- or double-stranded, but preferably be a double-stranded DNA. A nucleic acid may be isolated, or integrated into another nucleic acid molecule such as an expression vector or a chromosome of a eukaryotic host cell. A nucleic acid may be integrated into a vector through, such as, random integration, site-specific integration, a transposon system or the like.

The site-specific integration generally includes: site-specific integration mediated by gene editing tools, which include, but are not limited to transcription activator-like effector nuclease (TALEN): crispr, zinc-finger nuclease (ZFN) and clustered regulatory inter spaced short palindromic repeat (CRISPR); and site-specific integration based on DNA recombinases which include, but are not limited to e.g. Cre/LoxP, Flp/FRT, Bxb1 and phiC31/R4.

The transposon system generally includes, but not limited to, hAT transposon (Tol2), tc1 like transposon (e.g., Sleeping Beauty (SB) and Frog Prince (FP)), Piggy Bac, Helitrons transposon, DDE transposase mediated shear paste transposons (e.g., Tc1/Mariner, P element), tyrosine transposase transposon (e.g., Cryptons), Mavericks (i.e., Polinton), and the like.

An “isolated” nucleic acid molecule is one that is separated from other nucleic acid molecules present in the natural source of the nucleic acid. For example, in terms of genomic DNA, the term “isolated” includes nucleic acid molecules that are separated from the chromosomes with which the genomic DNA is naturally associated. Preferably, an “isolated” nucleic acid is free of sequences that naturally flank the nucleic acid (i.e., sequences located at the 5′ and 3′ ends of the nucleic acid) in the genomic DNA of the organism from which the nucleic acid is derived. In addition, an “isolated” nucleic acid molecule, such as a cDNA molecule, can be substantially free of other cellular material, or culture medium when produced by recombinant techniques, or chemical precursors or other chemicals when chemically synthesized.

The terms “recombinant vector” or “vector”, used interchangeably herein, refers to a nucleic acid molecule capable of transporting another nucleic acid to which it has been linked. One type of vector is a “plasmid”, which refers to a circular double stranded DNA loop into which additional DNA segments may be linked. Alternatively, a vector can be linear. Another type of vector is a viral vector, wherein additional DNA segments may be linked into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. The term “construct”, as used herein, may also refer to a vector, which can be improved by modulating the transcriptional regulatory elements (cis- or trans-regulatory or acting elements). Examples of commonly used transcriptional regulatory elements include promoters, enhancers, introns, non-coding regions, locus control regions, and the like.

Certain vectors are capable of directing the expression of genes to which they are operatively linked. An “expression vector” or “recombinant expression vector” is a nucleic acid molecule encoding a gene that is expressed in a host cell, and, furthermore, contains the necessary elements to control expression of the gene. Typically, an expression vector comprises a transcription promoter, a gene of interest, and a transcription terminator. Gene expression is usually placed under the control of a promoter, and such a gene is said to be “operatively linked to” the promoter. Similarly, a regulatory element and a core promoter are operatively linked if the regulatory element modulates the activity of the core promoter.

A nucleotide sequence is “operatively linked” when placed into a functional relationship with another nucleotide sequence. For example, a promoter or enhancer is operatively linked to a nucleotide sequence encoding a protein or polypeptide if expression of the protein or polypeptide is promoted or enhanced. In one embodiment, nucleotide sequences that are operatively linked are contiguous (e.g., in the case of signal sequences). Alternatively, nucleotide sequences that are operatively linked can be non-contiguous (e.g., in the case of enhancers). In one embodiment, the nucleic acid sequence encoding an antibody light or heavy chain constant region is operatively linked to the gene of interest, e.g., a heavy or light chain variable region.

Three different RNA polymerases are found in all eukaryotic cells, which, based on their individual sensitivities to α-Amanitin, are respectively referred as RNA polymerase I, RNA polymerase II, and RNA polymerase III. RNA polymerase I is located in the nucleolus and is used to synthesize rRNA precursors. RNA polymerase II is located in the nucleoplasm and is used to synthesize mRNA precursors and most snRNAs. RNA polymerase III is also located in the nucleoplasm and is used to synthesize 5S rRNA precursors, tRNA precursors, and other nuclear and cytoplasmic small RNA precursors.

As used herein, the term “promoter” includes any nucleic acid sequence sufficient to drive transcription in a eukaryotic cell, which is a DNA sequence recognized, bound, and transcription-initiated by the RNA polymerases. A promoter contains conserved sequences required for RNA polymerase specific binding and transcriptional initiation. There are three main types of promoters in eukaryotes, corresponding respectively to the three types of RNA polymerase mentioned above, namely type I, type II, and type III promoters. Promoters may comprise inducible promoters, repressible promoters and constitutive promoters. The inducible promoters require the addition of specific drugs to stimulate or inhibit its activity, such as tetracycline induced TRE/TRE3G expression. In the expression system, the most commonly used promoters are the constitutive promoters, which can maintain basically constant expression activity in a vast majority of cells.

In mammalian cells, the commonly used constitutive promoters include CMV (Cytomegalovirus) promoter; EF1α (Elongation factor-1α) promoter; EFS promoter (whitout introns due to the truncated form of EF1); CAG promoter (a chimeric promoter composed of cytomegalovirus enhancer and chicken β-actin promoter); CBh promoter (chicken β-Actin hybrid promoter); SFFV (spleen focus-forming virus) promoter; MSCV (Murine Stem Cell Virus) promoter; SV40 (simian vacuolar virus 40 sourced) promoter; mPGK (mouse phosphoglycerate kinase) promoter; hSYN (human synaptic protein) promoter; hPGK (human phosphoglycerate kinase) promoter; UBC (ubiquitin C) promoter; Human beta action (β-actin gene sourced) promoter; TRE (tetracycline responsive element) promoter; UAS (a drosophila promoter containing Gal4 binding site); Ac5 (a strong insect promoter derived from Actin 5c gene); Polyhedrin (a strong insect promoter derived from rod-shaped viruses); CaMKIIa (Ca2+/calmodulin dependent protein kinase II); and those artificial promoters commonly used in the art.

Promoters may be used with or without enhancers. Enhancer refers to a type of non-coding DNA cis-acting elements that activate or enhance gene transcription during the development of eukaryotes by binding transcription factors, cofactors, and chromatin complexes and thereby acting on promoters. Enhancers may be those naturally occurring and naturally related to nucleic acid sequences, and may be located downstream or upstream of the sequence to be regulated. Enhancers may also be recombinant or heterologous enhancers, i.e., those not naturally related to the nucleic acid sequences in their natural environment.

In some embodiments, the first expression cassette, second expression cassette and third expression cassette of the expression vector disclosed herein may respectively include the promoters and/or introns listed in the following table:

| | First expression | Second expression | Third expression |
|---|---|---|---|
| No. | cassette | cassette | cassette |
| 1 | CMV promoter | CMV promoter | SV40 promoter |
| 2 | CMV promoter | CMV promoter | UBC promoter |
| 3 | CMV promoter | CMV promoter | UBC promoter + |
| | | | (EF1α intron) |
| 4 | CMV promoter + | CMV promoter + | UBC promoter + |
| | (EF1α intron) | (EF1α intron) | (EF1α intron) |
| 5 | CMV promoter + | CMV promoter + | CMV promoter |
| | (EF1α intron) | (EF1α intron) | |
| 6 | CMV promoter + | CMV promoter + | CMV promoter + |
| | (EF1α intron) | (EF1α intron) | (EF1α intron) |
| 7 | CMV promoter + | CMV promoter + | SV40 promoter |
| | (EF1α intron) | (EF1α intron) | |
| 8 | CMV promoter + | CMV promoter + | SV40 promoter + |
| | (EF1α intron) | (EF1α intron) | (EF1α intron) |
| 9 | CMV promoter + | CMV promoter + | CMV promoter |
| | (CMV enhancer) | (CMV enhancer) | |
| 10 | EF1α promoter + | EF1α promoter + | SV40 promoter |
| | (EF1α intron) | (EF1α intron) | |
| 11 | EF1α promoter + | EF1α promoter + | SV40 promoter |
| | (CMV intron) | (CMV intron) | |
| 12 | CAG promoter + | CAG promoter + | SV40 promoter |
| | (CMV enhancer) | (CMVenhancer) | |

As used herein, the terms “upstream” and “downstream” are terms used to describe the relative orientation between two elements present in a nucleic acid sequence or vector. An element that is “upstream” of another is located in a position closer to the 5′ end of the sequence (i.e., closer to the end of a molecule that has a phosphate group attached to the 5′ carbon of the ribose or deoxyribose backbone if the molecule is linear) than the other element. An element is said to be “downstream” when it is located in a position closer to the 3′ end of the sequence (i.e., the end of the molecule that has a hydroxyl group attached to the 3′ carbon of the ribose or deoxyribose backbone in the linear molecule) when compared to the other element.

As used herein, the terms “ligation”, “fused” and “fusion” are interchangeable and refer to the connection of two or more elements or components by any means, such as chemical conjugation or recombination.

As used herein, the terms “transform” and “transfect” are interchangeable and refer to the uptake of exogenous DNA by cells through any practical method. When exogenous nucleic acids have been introduced into cell membrane, the cells are “transformed”. The uptake of a nucleic acid molecule leads to a stable transfector, independent of the procedure for uptake. The uptake procedures may include transfection (including electroporation), protoplast fusion, calcium phosphate precipitation, fusion with cells having envelope DNA, microinjection, and the like. The transformed cells are allowed to grow under conditions suitable for producing proteins of interest (e.g., antibody heavy and/or light chains in the embodiment) and subjected to assays to identify the encoded peptides of interest.

The term “IgM” refers to a type of immunoglobulins, which is a glycoprotein that forms polymers in which multiple immunoglobulins are covalently linked by disulfide bonds. IgM mainly exists as a pentamer, but also has the form of a hexamer. Thus, IgM can contain 10 or 12 antigen binding sites. The pentamer form of IgM generally contains a additional peptide called J chain, but can also be prepared without the J chain. The pentamer form of IgM has a molecular weight of approximately 970 kDa. Due to its polymerization, IgM has high affinity and is particularly effective in complement activation. Unlike IgG, a heavy chain in an IgM monomer is consisted of one variable domain and four constant domains.

The term “IgA” refers to another type of immunoglobulins, which is in a form of tetramer that contains two identical light chains (κ or λ) and two identical heavy chains (α). In the human body, there are two types of IgA isoforms, i.e., IgA1 and IgA2. Similar to IgG, IgA contains three constant domains (CA1-CA3, or Cα1-Cα3) and a hinge zone between Cα1 and Ca2 domains, in which “CA” and “Ca” can be used interchangeably. Both of the IgA isoforms have a “tail fragment” having 18 amino acids which is located at C-end of the Ca3 domain, and enables the formation of poly Ig. Serum IgA may be a monomer, but can also polymerize. In its secretory form, IgA contains 2-5 of the basic 4-chain units connected by a J chain, which may include a tail fragment and be associated by a secretory component. IgA antibodies can be further divided into IgA1 and IgA2 subclasses. As used herein, the term “IgA” antibody is intended to specifically encompass all subclasses, i.e., IgA1 and IgA2 antibodies, including dimer and multimers, with and without a secretory component, as well as fragments, preferably antigen-binding fragments, of such antibodies.

For purpose of clarifying the purpose, technical solution, and advantages of the present invention, the present invention is further described in details in connection with the following Examples. The particularly described Examples aim only to illustrate the present invention and are not intended to constitute any limitation to the present invention. In addition, in the following illustration, details of well-known structures and techniques have been omitted to avoid unnecessary confusion with the concepts disclosed herein. Such structure and technology have further been described in many publications.

### EXAMPLES

### Example 1. Construction of IgM Expression Vectors Comprising mCMV Promoter

In this Example, a vector for expressing IgM was constructed by means of mCMV promoter.

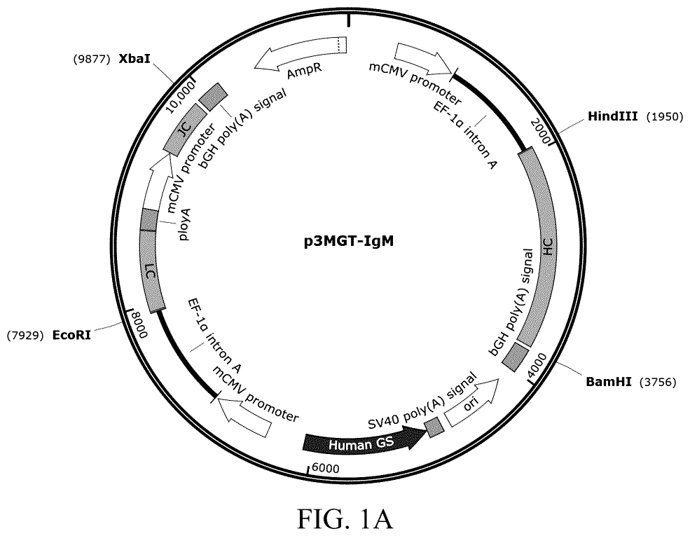

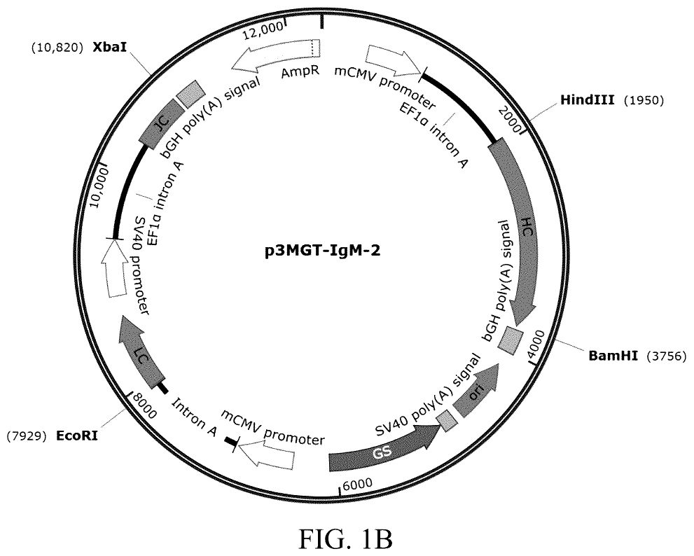

For the single-vector expression system, a vector p3MGT-IgM was used (FIG. 1A). In brief, mCMV promoter (SEQ ID NO:1) and intron A of EF-1α (SEQ ID NO:5) were operatively linked upstream of the heavy chain (HC, SEQ ID NO: 6) and light chain (LC, SEQ ID NO: 7) coding regions of anti-CD20 IgM, respectively. mCMV promoter (SEQ ID NO:1) was operatively linked upstream of the J chain (JC, SEQ ID NO: 8) coding region. The constructed vector was named as CD20 p3MGT-IgM-1.

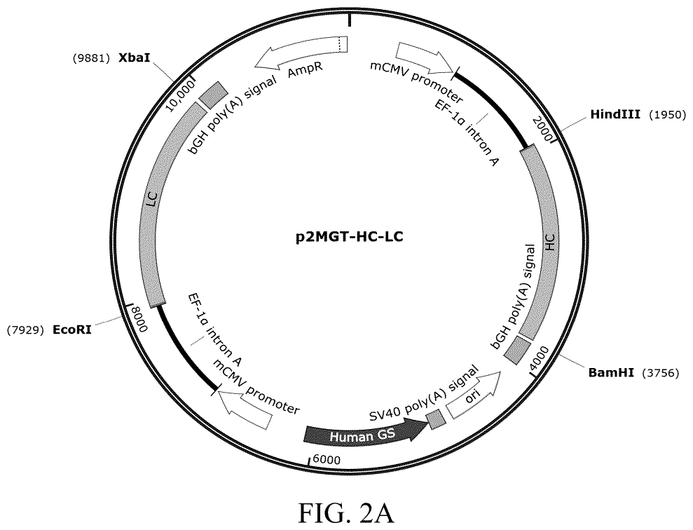

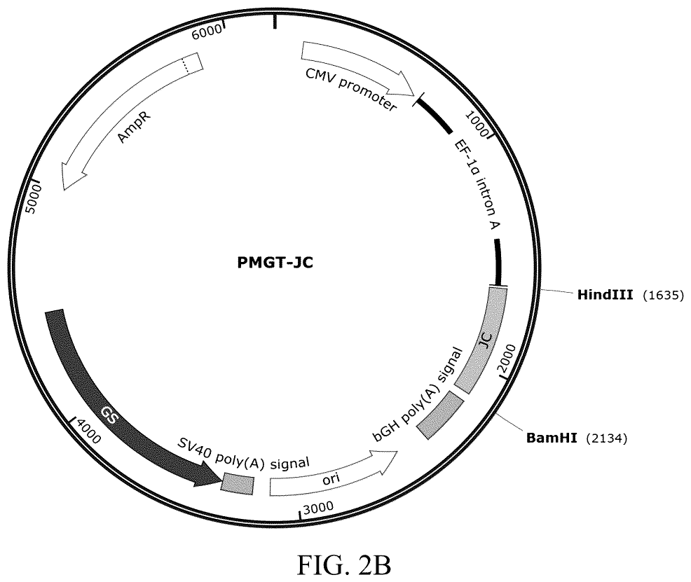

For the double-vector expression system, vectors p2MGT-HC-LC and pMGT-JC expression system (FIGS. 2A and 2B) were used. In brief, the HC- and LC-coding regions of anti-CD20 IgM were located in the same vector p2MGT-HC-LC. mCMV promoter and intron A of EF-1α were operatively linked upstream of both the HC- and LC-coding regions. The JC-coding region was located in vector pMGT-JC, and mCMV promoter was operatively linked upstream thereof. The constructed vectors were named as CD20 p2MGT-HC-LC and CD20 pMGT-JC, respectively.

In these vectors, the HC of anti-CD20 IgM has an amino acid sequence as shown in SEQ ID NO: 6, the LC of anti-CD20 IgM has an amino acid sequence as shown in SEQ ID NO: 7, and the J chain of anti-CD20 IgM has an amino acid sequence as shown in SEQ ID NO: 8.

### Example 2. Expression of IgM

In this Example, the single- and double-vector expression systems constructed in Example 1 are respectively used for expressing anti-CD20 IgM.

1. Thawing and passage of host cells CHOZN CHOK1 cells were thawed in EX-CELL® CD CHO Fusion culture medium (Merck, containing 4 mm L-glutamine) and allowed to recover by passaging at least three generations in EX-CELL® CD CHO Fusion culture medium (Merck, containing 4 mm L-glutamine). The cells were passaged once every 3 days, and seeded at a viable cell density of 0.3-0.4×10⁶ cells/mL.

Cell culturing conditions: 180 rpm (cell culture tubes), 5% CO₂, 85% humidity.

Cell counting: COUNSTAR cell counter, VI-CELL cell counter.

2. Transfection

After the recovery of the host cells and at least 3 passages, a volume of cells in logarithmic growth phase was transfered into a 50-mL cell culture tube and centrifuged at 1000 rpm for 5 min. The supernatant was discarded, and the pellet was resuspended with a corresponding volume of fresh Hyclone HyCell TransFx-C culture medium (containing 4 mm L-glutamine), adjusted to a viable cell density of 3.0×10⁶ cells/mL for transfection. 1 mg/mL PEI was used for the transfection, at a 1:3 ratio of DNA to PEI, and 2-3 μg plasm ids per 10⁶ cells. The cells were transfected with CD20 p3MGT-IgM-1, or co-transfected with CD20 p2MGT-HC-LC and CD20 pMGT-JC (at a ratio of 5:1 or 4:1, respectively).

After transfection, the cells were cultured in a shaker for 2 days. Cell culture conditions: 37° C., 180 rpm, 5% CO₂, 85% humidity.

3. Cell Pool Screening

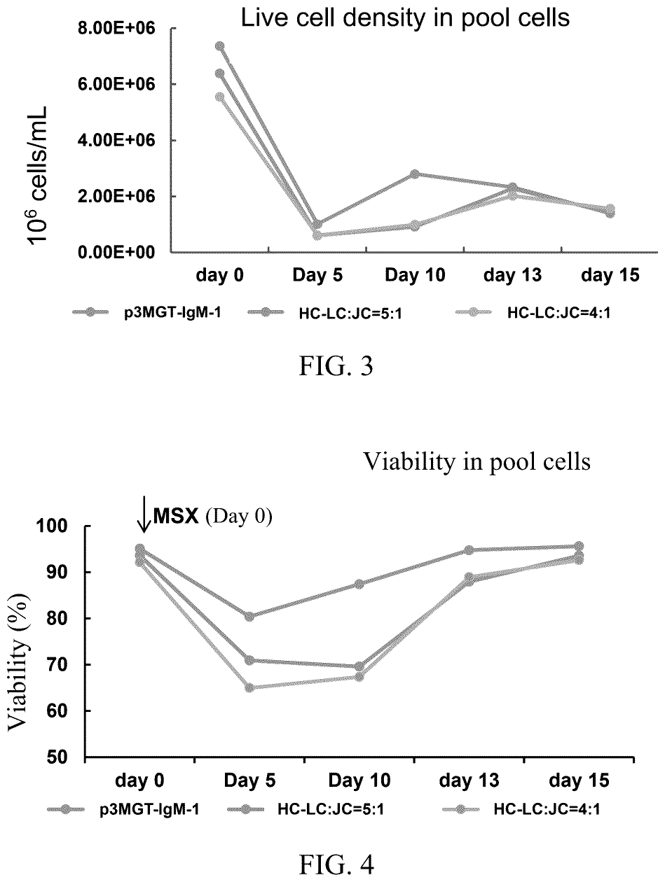

48h after transfection, the cells were subjected to a pressure screening with EX-CELL Advanced CHO Fed-batch culture medium (Sigma) containing 25 μm methionine sulfoximine (msx). The viable cell density (VCD) and viability rates were detected according to conventional experimental procedures by means of COUNSTAR cell counter. The cells were passaged every 3-4 days and pressure screened, until the viability rates of the cells were restored to 90% or more. The results are shown in FIG. 3. It can be seen that, a higher viable cell density are achieved when HC, LC and JC are simultaneously expressed in one plasm id p3MGT-CD20.

Meanwhile, the cell viability rate was detected, and the results are shown in FIG. 4. From the results in FIG. 4, it can be seen that the cell viability rates obviously restores faster in the case that HC, LC, and JC are simultaneously expressed in one plasm id p3MGT CD20, under MSX pressure screening.

4. Fed-Batch Culture of Cell Pools

The restored cells were seeded at a cell density of 0.5×10⁶ cells/m L. From the third day, fresh medium was added and continued through the process of cultivation.

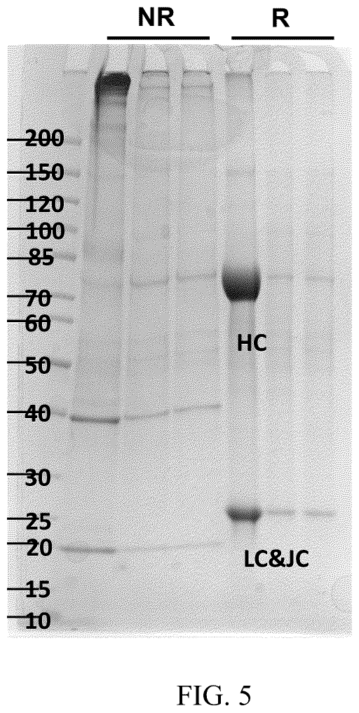

The cell culture supernatants were added with SDS-PAGE loading buffer, heated at 100° C. for 6 minutes, and detected with reduced (r) and non-reduced (nr) SDS-PAGE respectively for IgM expression. The results are shown in FIG. 5. From FIG. 5, it can be seen that the IgM expression level is significantly higher in the case that HC, LC, and JC are simultaneously expressed in plasmid CD20 p3MGT-IgM-1, than that in cells co-transfrected with plasm ids CD20 p2MGT-HC-LC and CD20 pMGT-JC at a ratio of 5:1 or 4:1.

### Example 3. Identification of the Expression Levels of Various Chains

The anti-CD20 IgM antibody obtained in Example 2 by expressing HC, LC, and JC using plasmid CD20 p3MGT-IgM-1 was subjected to IgM purification and mass spectrometry assay, in order to detect the de-N-glycosylation reduced mass.

The results are shown in the following Table 1. It can be seen that the molecular weights of the obtained HC, LC and JC are respectively identical to their theoretical molecular weights.

TABLE 1
Results of Molecular Weights of various chains of the obtained
anti-CD20 IgM antibody after Deglycosylation and Reduction

| | Found | Theoretical | |
|---|---|---|---|
| Target | molecular | molecular | Mass error |
| component/modification | weight (Da) | weight (Da)* | (Da) |
| JC: pyroQ | 15577.35 | 15577.32 | 0.03 |
| LC: pyroQ | 23038.49 | 23039.43 | −0.94 |
| HC: pyroQ | 62608.96 | 62609.64 | −0.68 |

### Example 4. Construction of IgM Expression Vectors Containing SV40 Promoters

In order to verify the effects of different promoters and enhancers on the expression of IgM antibodies, the following expression vectors expressing anti-CD20 IgM antibodies were constructed in this Example. The vector p3MGT-IgM-2 was used (FIG. 1B). In brief, mCMV promoter (SEQ ID NO:1) and intron A of EF-1a (SEQ ID NO:5) were operatively linked upstream of the heavy chain (HC, SEQ ID NO: 6) and light chain (LC, SEQ ID NO: 7) coding regions of anti-CD20 IgM. SV40 promoter (SEQ ID NO:12) and intron A of EF-1α (SEQ ID NO:5) were operatively linked upstream of J chain (JC, SEQ ID NO: 8) coding region. The constructed vector was named as CD20 p3MGT-IgM-2.

The two expression systems of Example 1 and the expression vector constructed in this Example were used for the expression of IgM.

1. Thawing and Passage of Host Cells

CHOZN CHOK1 cells were thawed in EX-CELL® CD CHO Fusion culture medium (Merck, containing 4 mm I-glutamine) and allowed to recover by passaging at least three generations in EX-CELLO CD CHO Fusion culture medium (Merck, containing 4 mm L-glutamine). The cells were passaged once every 3 days, and seeded at a viable cell density of 0.3-0.4×10⁶ cells/m L.

Cultivation conditions: 180 rpm (cell culture tubes), 5% CO₂, 85% humidity.

Cell counting: COUNSTAR cell counter, VI-CELL cell counter.

2. Transfection

After the recovery of the host cells and at least 3 passage, a volume of cells in logarithmic growth phase was transfered into a 50-mL cell culture tube and centrifuged at 1000 rpm for 5 min. The supernatant was discarded, and the pellet was resuspended with a corresponding volume of fresh Hyclone HyCell TransFx-C culture medium (containing 4 mm L-glutamine), adjusted to a viable cell density of 3.0×10⁶ cells/mL for transfection. 1 mg/mL PEI was used for the transfection, at a 1:3 ratio of DNA to PEI, and 2-3 μg plasm id per 10⁶ cells. The cells were respectively transfected with CD20 p3MGT-IgM-1, CD20 p3MGT-IgM-2, or co-transfected with CD20 p2MGT-HC-LC and CD20 pMGT-JC (at a ratio of 5:1 or 4:1, respectively).

After transfection, the cells were cultured in a shaker for 48h. Cell culture conditions: 37° C., 180 rpm, 5% CO₂, 85% humidity.

3. Cell Pool Screening

48h after transfection, the transfected cells were subjected to a pressure screening with EX-CELL Advanced CHO Fed-batch culture medium (SIGMA) containing 25 μm methionine sulfoximine (msx). The viable cell density (VCD) and viability rates were detected according to conventional experimental procedures by means of COUNSTAR cell counter. The cells were passaged every 3-4 days and pressure screened, until the viability rates of the cells were restored to 90% or more.

4. Fed-Batch Culture of Cell Pool

The restored cell pools were seeded at a cell density of 0.5×10⁶ cells/m L. From the third day, fresh medium was added and continued through the process of cultivation.

The cell culture supernatants were added with SDS-PAGE loading buffer, heated at 100° C. for 6 minutes, and detected with non-denatured PAGE for IgM expression. From FIG. 6, it can be seen that the expression of IgM is obviously observed in the case that HC, LC, and JC of anti-CD20 are simultaneously expressed by using the single expression vectors CD20 p3MGT-IgM-1 (Lane 3) and CD20 p3MGT-IgM-2 (Lane 4). While no IgM multimer is observed for the co-transfrection group of CD20 p2MGT-HC-LC and CD20 pMGT-JC at either ratio of 5:1 or 4:1.

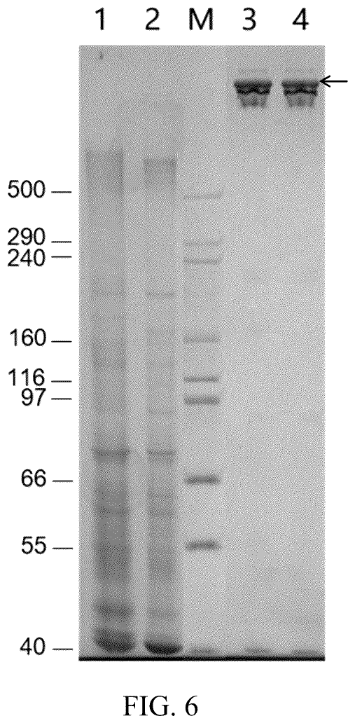

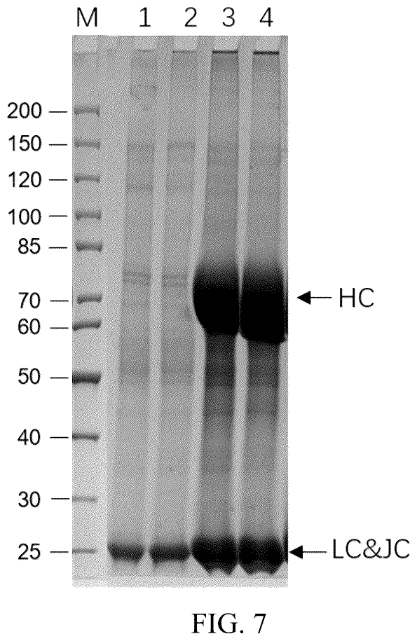

Subsequently, the expressions of anti-CD20 IgM of different expression systems were compared by using reduced SDS-PAGE. In brief, the cell culture supernatants were added with SDS-PAGE loading buffer, heated at 100° C. for 6 minutes, and detected with reduced SDS-PAGE for the expression of IgM. The results are shown in FIG. 7. From FIG. 7, it can be seen that the expression levels of the IgM heavy chain are significantly higher in all the cases that HC, LC, and JC of anti-CD20 are simultaneously expressed in both of the single expression vectors CD20 p3MGT-IgM-1 and CD20 p3MGT-IgM-2, even under the control of different promoters and introns, than those in cells co-transfected with CD20 p2MGT-HC-LC and CD20 pMGT-JC in either ratio of 5:1 or 4:1. Insufficient heavy chain expression may be the main reason for the low yield of IgM co-transfected with the two plasm ids.

Further, IgM affinity chromatography was used to determine the titers of anti-CD20 IgM obtained by using the expression vector CD20 p3MGT-IgM-1 and CD20 p3MGT-IgM-2, as well as the two-vector system CD20 p2MGT-HC-LC and CD20 pMGT-JC in a ratio of 5:1 or 4:1. In brief, the titers of anti-CD20 IgM were determined by means of high-performance liquid chromatography having a POROS CaptureSelect IgM affinity column (Thermo Fisher Scientific, 4469164) Mobile phase A (50 mM PB, 300 mM NaCl, pH7.0) for loading, mobile phase B (50 mM PB, 300 mM NaCl, pH2.2) for elution. The concentrations of the proteins were determined using a UV detector. The results are shown in Table 2 below.

TABLE 2
High-performance liquid chromatography results
of anti-CD20 IgM from cell pools.

| Sample | Titer (g/L) |
|---|---|
| CD20 p3MGT-IgM-1 | 5.6 |
| CD20 p3MGT-IgM-2 | 5.44 |
| CD20 p2MGT-HC-LC:CD20 pMGT-JC = 5:1 | 0.02 |
| CD20 p2MGT-HC-LC:CD20 pMGT-JC = 4:1 | 0.02 |

The results of the high-performance liquid chromatography shown in table 2 are consistent with the PAGE results as shown in FIGS. 6 and 7. It may be achieved that the light and heavy chains are expressed at a higher level of expression than that of J chain by adjusting the relative transcription-initiating capabilities of the promoters for the light, heavy and J chains, when HC, LC, and JC of anti-CD20 are simultaneously expressed in a single expression vector CD20 p3MGT-IgM-1 or CD20 p3MGT-IgM-2. In such case, there should be no significant difference between the levels of the expressions of the light and heavy chains. IgM can have a high yield, which may be in a range of 5.4-5.6 g/L in cell pools and be significantly higher than those in cell pools co-transfected with CD20 p2MGT-HC-LC and CD20 pMGT-JC, in either ratio of 5:1 or 4:1.

The expressions of HC, LC, and JC of anti-CD20 IgM were detected by using real time fluorescence quantitative PCR (qPCR) for the cells transfected with the various expression vectors constructed in Examples 2 and 4. In brief, RNA was isolated from the cell samples obtained from the fed-batch culture by using NucleoSpin® RNA Plus kit. Then, the cDNA was obtained based on the isolated RNA through reverse transcription with PrimeScript™ IV first strand cDNA synthesis kit, and was further used as the template for qPCR detection. The primers used for the qPCR detection were as follows:

IgM-HC Primer_F:
(SEQ ID NO: 13)
5′-TTTCTGCCTGACAGCATCAC-3′,
IgM-HC Primer_R:
(SEQ ID NO: 14)
5′-GAGGCACGTTCTTCTCCTTG-3′,
IgM-LC Primer_F:
(SEQ ID NO: 15)
5′-CTACAGCCTGACCATCAGCA-3′,
IgM-LC Primer_R:
(SEQ ID NO: 16)
5′-ACTGCACCTTGGCCTCTCTA-3′,
IgM-JC Primer_F:
(SEQ ID NO: 17)
5′-CCAAGAGGACGAGAGAATCG-3′,
IgM-JC Primer_R:
(SEQ ID NO: 18)
5′-GCTTGTAGGGTCGCTGATGT-3′,

The nternal reference primers were as follows:

EF1α-F:
(SEQ ID NO: 19)
5′-TGCCACCAACTCGTCCCACT-3′,
EF1α-R:
(SEQ ID NO: 20)
5′-TCCCACAGGAACAGTCCCAATAC-3′.

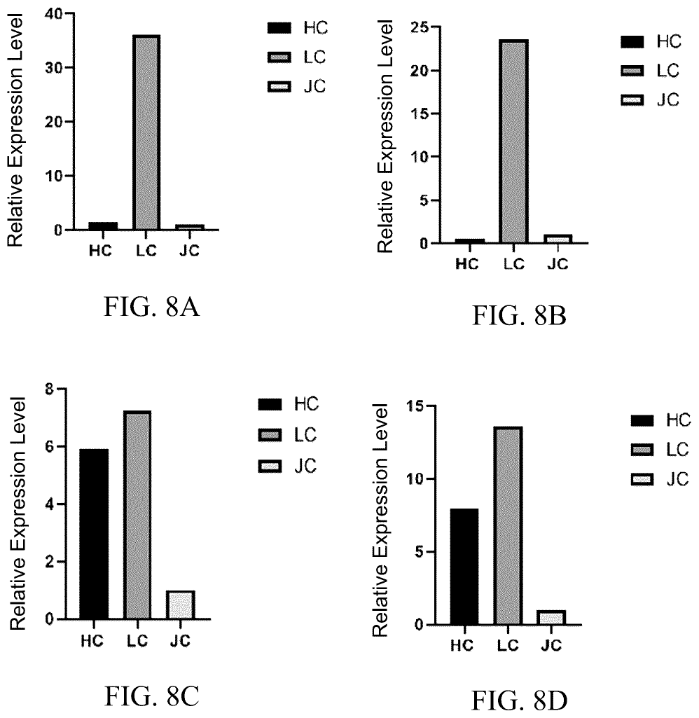

The results of the transcription levels of HC, LC, and JC are shown in FIGS. 8A-8D. As shown in FIGS. 8C (p3MGT-IgM-1) and 8D (p3MGT-IgM-2), the proportions of HC, LC, and JC are more balanced at the mRNA transcription level, and both of HC and LC have higher transcription levels than that of JC, when HC, LC, and JC of anti-CD20 are simultaneously expressed in the single expression vector. While in the co-transfection groups of CD20 p2MGT-HC-LC and CD20 pMGT-JC at ratios of 5:1 (FIG. 8A) and 4:1 (FIG. 8B), HC is expressed at much lower levels, which are consistent with the results as shown in FIGS. 5 and 7. In FIG. 8A, the ratio of HC:LC:JC is about 1.54:36:1 at the transcription level. In FIG. 8B, the ratio of HC:LC:JC is about 0.59:23.6:1 at the transcription level. In FIG. 8C, the ratio of HC:LC:JC is about 5.9:7.25:1 at the transcription level. In FIG. 8D, the ratio of HC:LC:JC I is about 7.94:13.61:1 at the transcription level.

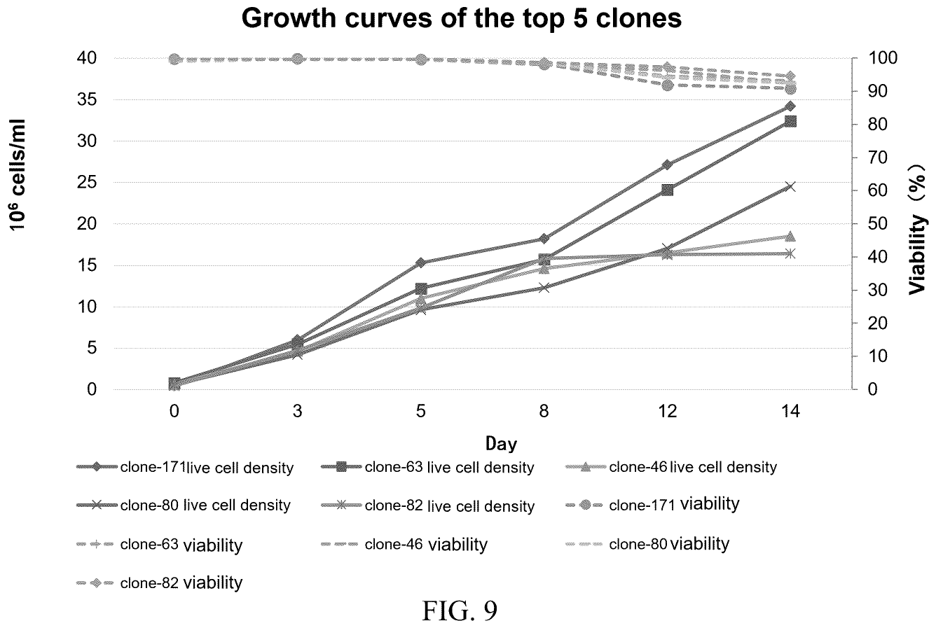

Monoclonal colonies containing CD20 p3MGT-IgM-2 were plated and cultivated for three weeks. The cells were amplified to 24-well plates, 6-well plates and shake tubes sequentially. After fully suspended adaptation, 30 clones were picked and seeded at a cell density of 0.5×10⁶ cells/mL for fed-batch culture. From the third day, fresh medium was added and continued through the process of cultivation. The titers of IgM in the supernatants were determined on the fourteenth day by IgM affinity chromatography. The growth curves of top 5 clones are shown in FIG. 9 and their production yields are shown in Table 3. As can be seen, the highest yield can reach about 8.34 g/L.

TABLE 3
Analysis results of high-performance liquid chromatography
of top 5 clones expressing anti-CD20 IgM.

| Clone No. | Titer (g/L) |
|---|---|
| Clone-171 | 8.34 |
| Clone-63 | 7.91 |
| Clone-46 | 7.27 |
| Clone-80 | 6.85 |
| Clone-82 | 6.33 |

### Example 5. Construction of Expression Vectors of Anti-Her2 IgM Antibodies and Expression Thereof

In this Example, the expression vectors of anti-Her2 IgM antibody were constructed. For the single vector expression system, a vector p3MGT-IgM-1 (FIG. 1A) was used. In brief, the mCMV promoter (SEQ ID NO:1) and intron A of EF-1α (SEQ ID NO:5) were operatively linked upstream of the heavy chain (HC, SEQ ID NO: 9) and light chain (LC, SEQ ID NO: 10) coding regions of anti-Her2 IgM. The mCMV promoter (SEQ ID NO:1) was operatively linked upstream of J chain (JC, SEQ ID NO: 11) coding region. The constructed vector was named as Her2 p3MGT-IgM-1.

For the double-vector expression system, vectors p2MGT-HC-LC and pMGT-JC were used. In brief, the HC (heavy chain) and LC (light chain) coding regions of anti-Her2 IgM were located in the same vector p2MGT-HC-LC. The mCMV promoter and intron A of EF-1α were operatively linked upstream of both the HC- and LC-coding regions. JC (J chain) coding region was located in vector pMGT-JC. and mCMV promoter was operatively linked upstream thereof. The constructed vectors were named as Her2 p2MGT-HC-LC and Her2 pMGT-JC, respectively.

The heavy chain of anti-Her2 IgM has an amino acid sequence as shown in SEQ ID NO: 9, the light chain of anti-Her2 IgM has an amino acid sequence as shown in SEQ ID NO: 10, and the J chain of anti-Her2 IgM has an amino acid sequence as shown in SEQ ID NO: 11.

Expression of the anti-Her2 IgM antibody:

In this Example, the anti-Her2 IgM antibody was expressed in two manners.

1. Thawing and Passage of Host Cells

CHOZN CHOK1 cells were thawed in EX-CELL® CD CHO Fusion culture medium (Merck, containing 4 mm L-glutamine) and allowed to recover by passaging at least three times in EX-CELLO CD CHO Fusion culture medium (Merck, containing 4 mm L-glutamine). The cells were passaged once every 3 days, and seeded at a viable cell density of 0.3-0.4×10⁶ cells/mL.

Cultivation conditions: 180 rpm (cell culture tubes), 5% CO₂, 85% humidity.

2. Transfection

After the recovery of the host cells and at least 3 passages, a volume of cells in logarithmic growth phase was transfered into a 50-mL cell culture tube and centrifuged at 1000 rpm for 5 min. The supernatant was discarded, and the pellet was resuspended with a corresponding volume of fresh Hyclone HyCell TransFx-C culture medium (containing 4 mm L-glutamine), adjusted to a viable cell density of 3.0×10⁶ cells/mL for transfection. 1 mg/ml PEI was used for the transfection, at a 1:3 ratio of DNA to PEI, and 2-3 μg plasm ids per 10⁶ cells. The cells were transfected with Her2 p3MGT-IgM-1, or co-transfected with Her2 p2MGT-HC-LC and Her2 pMGT-JC at a ratio of 5:1 or 4:1, respectively.

After transfection, the cells were cultured in a shaker for 2 days. Cell culture conditions: 37° C., 180 rpm, 5% CO₂, 85% humidity.

3. Cell Pool Screening

After transfection for 48h, the cells were subjected to a pressure screening with EX-CELL Advanced CHO Fed-batch culture medium (SIGMA) containing 25 μm methionine sulfoximine (msx). The viable cell density (VCD) and viability rates were determined according to conventional experimental procedure. The cells were passaged every 3-4 days and pressure screened, until the viability rates were restored to 90% or more.

4. Fed-Batch Culture of Cell Pools

The restored cells were seeded at a cell density of 0.5×10⁶ cells/mL for fed-batch culture. From the third day, fresh medium was added and continued through the process of cultivation.

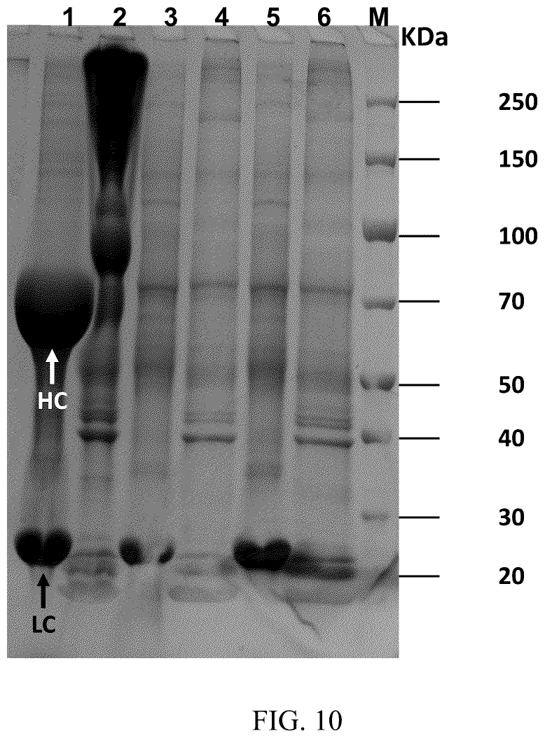

The cell culture supernatants were added with SDS-PAGE loading buffer, heated at 100° C. for 6 minutes, and detected with reduced (r) and non-reduced (nr) PAGE for IgM expression. The results are shown in FIG. 10.

From FIG. 10, it can be seen that the expression level of IgM is significantly higher in the case that HC, LC, and JC of anti-Her2 are simultaneously expressed in Her2 p3MGT-IgM-1 plasmid, than that in the cells co-transfected with Her2 p2MGT-HC-JC and Her2 pMGT-JC in a ratio of either 5:1 or 4:1.

The present disclosure is not limited to the specific embodiments illustrated above.

Technical solutions resulted from any modification on basis of the above disclosure should be within the scope of protection of this disclosure.

What is claimed:

1. An expression vector, comprising:
a first expression cassette having a first transcriptional regulatory element and an immunoglobulin heavy chain-coding region which is operatively linked to the first transcriptional regulatory element;
a second expression cassette having a second transcriptional regulatory element and an immunoglobulin light chain-coding region which is operatively linked to the second transcriptional regulatory element; and,
a third expression cassette having a third transcriptional regulatory element and an immunoglobulin J chain-coding region operatively linked to the third transcriptional regulatory element,
wherein the expression levels of the first expression cassette and/or the second expression cassette are not less than the expression level of the third expression cassette, or the first transcriptional regulatory element and/or the second transcriptional regulatory element has a capability of initiating transcription no less than that of the third transcriptional regulatory element, and
wherein the first expression cassette, the second expression cassette and the third expression cassette are located within the same vector.

2. The expression vector according to claim 1, wherein the first and/or second transcriptional regulatory element have transcription-initiating capability higher than or equal to that of the third transcriptional regulatory element,
preferably, the first transcriptional regulatory element comprises a first promoter, which more preferably is a promoter for expression in a mammalian cell;
preferably, the second transcriptional regulatory element comprises a second promoter, which more preferably is a promoter for expression in a mammalian cell;
preferably, the third transcriptional regulatory element comprises a third promoter, which more preferably is a promoter for expression in a mammalian cell.

3. The expression vector according to claim 2, wherein the first promoter, the second promoter and/or the third promoter are one or more selected from a group consisting of CMV promoter, EF1α promoter, CAG promoter, CBh promoter, SFFV promoter, EFS promoter, MSCV promoter, SV40 promoter, mPGK promoter, hPGK promoter, UBC promoter, human beta actin promoter, TRE promoter, UAS promoter, Ac5 promoter, Polyhedrin promoter, CaMKIIa promoter and an artificial promoter,
preferably, the first promoter and/or the second promoter is one or more selected from a group consisting of CMV promoter, EF1α promoter, CAG promoter, CBh promoter, human beta actin promoter, SFFV promoter and an artificial promoter,
preferably, the third promoter is one or more selected from a group consisting of CMV promoter, EF1α promoter, CAG promoter, CBh promoter, SFFV promoter, EFS promoter, MSCV promoter, SV40 promoter, mPGK promoter, hPGK promoter, UBC promoter, human beta actin promoter, TRE promoter, UAS promoter, Ac5 promoter, Polyhedrin promoter and CaMKIIa promoter, more preferably the third promoter is one or more selected from a group consisting of CMV promoter, SV40 promoter, and UBC promoter.

4. The expression vector according to claim 1, wherein the first transcriptional regulatory element is located upstream of the immunoglobulin heavy chain-coding region;
and/or the second transcriptional regulatory element is located upstream of the immunoglobulin light chain-coding region; and/or
the third transcriptional regulatory element is located upstream of the immunoglobulin J chain-coding region.

5. The expression vector according to claim 2, wherein the first transcriptional regulatory element and/or the second transcriptional regulatory element further comprise one or more intron,
preferably, the intron is located downstream of the first promoter and/or second promoter,
more preferably, the intron is selected from a group consisting of human β-globin intron, CMV intron or EF1α intron.

6. The expression vector according to claim 1, wherein the immunoglobulin comprises IgM and/or IgA, preferably is IgM.

7. The expression vector according to claim 1, wherein the ratio of the expression level of the immunoglobulin heavy and/or light chain to the expression level of the J chain is ≥1,
preferably, the ratio of the expression levels of the immunoglobulin heavy chain, the immunoglobulin light chain, and the J chain is (4-20): (4-20): (1-4).

8. The expression vector according to claim 1, wherein the ratio of the expression level of the heavy chain to the expression level of the light chain is in a range of 0.1 to 10;
preferably, the ratio of the expression level of the heavy chain to the expression level of the light chain is in a range of 0.125 to 8.0;
preferably, the ratio of the expression level of the heavy chain to the expression level of the light chain is in a range of 0.2 to 5.0;
preferably, the ratio of the expression level of the heavy chain to the expression level of the light chain is in a range of 0.3 to 3.0;
preferably, the ratio of the expression level of the heavy chain to the expression level of the light chain is in a range of 0.4 to 2.0.

9. A host cell comprising the expression vector as defined in claim 1.

10. The host cell according to claim 9, wherein the host cell is selected from a group consisting of Chinese hamster ovary (CHO) cell, Chinese hamster fibroblast, human cervical cancer cell, monkey kidney cell, murine fibroblast, murine myeloma cell, hamster kidney cell, murine L cell, human lymphocyte and human embryonic kidney (HEK) cell, preferably is CHO cell or HEK293 cell.

11. A method for producing an immunoglobulin, comprising the following steps:
culturing the host cell as defined in claim 8; and
harvesting the immunoglobulin.

12. An immunoglobulin produced by using the expression vector as defined in claim 1, wherein the immunoglobulin comprises IgM and/or IgA, preferably is IgM.

13. Use of the expression vector as defined in claim 1 for increasing an expression level of an immunoglobulin, wherein the ratio of the expression level of an immunoglobulin heavy and/or light chain to the expression level of a J chain is greater than 1, and the ratio of the expression level of the heavy chain to the expression level of the light chain is in a range of 0.1 to 10;
preferably, the ratio of the expression level of the heavy chain to the expression level of the light chain is in a range of 0.125 to 8.0;
preferably, the ratio of the expression level of the heavy chain to the expression level of the light chain is in a range of 0.2 to 5.0;
preferably, the ratio of the expression level of the heavy chain to the expression level of the light chain is in a range of 0.3 to 3.0;
preferably, the ratio of the expression level of the heavy chain to the expression level of the light chain is in a range of 0.4 to 2.0;
preferably, the immunoglobulin comprises IgM and/or IgA, more preferably is IgM.


SEQUENCE LISTING (20 sequences)

SEQ ID NO: 1 - DNA, 532 bp, synthetic construct
  source 1..532: mol_type = other DNA
gatatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcactcattctattggctgagctgcgttctacgtgggtataagaggcgcgaccagcgtcggtaccgtcgcagtcttcggtctgaccaccgtagaacgcagatc

SEQ ID NO: 2 - DNA, 1193 bp, synthetic construct
  source 1..1193: mol_type = other DNA
acgcgttccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagaattc

SEQ ID NO: 3 - DNA, 137 bp, synthetic construct
  source 1..137: mol_type = other DNA
caggtaagtatcaaggttacaagacaggtttaaggagaccaatagaaactgggcttgtcgagacagagacgactcttgcgtttctgataggcacctattggtcttactgacatccactttgcctttctctccacagg

SEQ ID NO: 4 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 5 - DNA, 523 bp, synthetic construct
  source 1..523: mol_type = other DNA
gcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 6 - protein, 574 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 7 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 9 - protein, 575 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCYLC

SEQ ID NO: 10 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 137 aa, synthetic construct; identical to SEQ ID NO: 8

SEQ ID NO: 12 - DNA, 330 bp, synthetic construct
  source 1..330: mol_type = other DNA
gtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaa

SEQ ID NO: 13 - DNA, 20 bp, synthetic construct
  source 1..20: mol_type = other DNA
tttctgcctgacagcatcac

SEQ ID NO: 14 - DNA, 20 bp, synthetic construct
  source 1..20: mol_type = other DNA
gaggcacgttcttctccttg

SEQ ID NO: 15 - DNA, 20 bp, synthetic construct
  source 1..20: mol_type = other DNA
ctacagcctgaccatcagca

SEQ ID NO: 16 - DNA, 20 bp, synthetic construct
  source 1..20: mol_type = other DNA
actgcaccttggcctctcta

SEQ ID NO: 17 - DNA, 20 bp, synthetic construct
  source 1..20: mol_type = other DNA
ccaagaggacgagagaatcg

SEQ ID NO: 18 - DNA, 20 bp, synthetic construct
  source 1..20: mol_type = other DNA
gcttgtagggtcgctgatgt

SEQ ID NO: 19 - DNA, 20 bp, synthetic construct
  source 1..20: mol_type = other DNA
tgccaccaactcgtcccact

SEQ ID NO: 20 - DNA, 23 bp, synthetic construct
  source 1..23: mol_type = other DNA
tcccacaggaacagtcccaatac